SEQUENCE LISTING (38 sequences)

SEQ ID NO: 1 - DNA, 23 bp, Homo sapiens
gagccaatcagtagccagcatct

SEQ ID NO: 2 - DNA, 24 bp, Homo sapiens
gttcccccatttctatttctcgtt

SEQ ID NO: 3 - DNA, 21 bp, Homo sapiens
ctccctgaagtaagcaaagac

SEQ ID NO: 4 - DNA, 19 bp, Homo sapiens
ccatgtggcctgaaagtag

SEQ ID NO: 5 - DNA, 22 bp, Homo sapiens
ggcagatacatcccattccttc

SEQ ID NO: 6 - DNA, 24 bp, Homo sapiens
ggttgcttcatctccttggtcctt

SEQ ID NO: 7 - DNA, 19 bp, Homo sapiens
caggggcctttttgtgctc

SEQ ID NO: 8 - DNA, 22 bp, Homo sapiens
catggtggactgcgtgtaagat

SEQ ID NO: 9 - DNA, 21 bp, Homo sapiens
aggagggaggtattcgtaagg

SEQ ID NO: 10 - DNA, 21 bp, Homo sapiens
ttcaagggtttctccaatctg

SEQ ID NO: 11 - DNA, 22 bp, Homo sapiens
tggttacagcacaaaatgatgg

SEQ ID NO: 12 - DNA, 24 bp, Homo sapiens
ttgcctaaaatgagtgatgtgttg

SEQ ID NO: 13 - DNA, 24 bp, Homo sapiens
gccgccctaaaagcacactcatcc

SEQ ID NO: 14 - DNA, 24 bp, Homo sapiens
ttcccttgtcctctcctcccatcc

SEQ ID NO: 15 - DNA, 22 bp, Homo sapiens
tgcagccaaatcttcaactacc

SEQ ID NO: 16 - DNA, 22 bp, Homo sapiens
aaacctttcacacccacacctt

SEQ ID NO: 17 - DNA, 18 bp, Homo sapiens
agccttggccctgttgta

SEQ ID NO: 18 - DNA, 21 bp, Homo sapiens
tgcccatatctgctcactgta

SEQ ID NO: 19 - DNA, 22 bp, Homo sapiens
gccccgcctttcttcctgctca

SEQ ID NO: 20 - DNA, 24 bp, Homo sapiens
ccacctactcccctttcccacgac

SEQ ID NO: 21 - DNA, 24 bp, Homo sapiens
ctctgccttttgcttggatacata

SEQ ID NO: 22 - DNA, 22 bp, Homo sapiens
cacggcgtctgagatttgagtt

SEQ ID NO: 23 - DNA, 18 bp, Homo sapiens
ccgtgcttgtccctgtgg

SEQ ID NO: 24 - DNA, 21 bp, Homo sapiens
ctgtgcctcaagagactgttc

SEQ ID NO: 25 - DNA, 20 bp, Homo sapiens
agtttatgattccacctaca

SEQ ID NO: 26 - DNA, 20 bp, Homo sapiens
ttcaacatcctaaacataaa

SEQ ID NO: 27 - DNA, 24 bp, Homo sapiens
gtggccgagaaagcagggtagacg

SEQ ID NO: 28 - DNA, 24 bp, Homo sapiens
caggcgcaagagcagcatccacat

SEQ ID NO: 29 - DNA, 24 bp, Homo sapiens
gatccctgccctcctgctgactat

SEQ ID NO: 30 - DNA, 24 bp, Homo sapiens
aggccaaccatgatgtgctgaaac

SEQ ID NO: 31 - DNA, 19 bp, Homo sapiens
ccggatcctgggtggtctg

SEQ ID NO: 32 - DNA, 18 bp, Homo sapiens
ccgccgggtttgattgat

SEQ ID NO: 33 - DNA, 22 bp, Homo sapiens
gactttccttcctccatctcca

SEQ ID NO: 34 - DNA, 19 bp, Homo sapiens
ggccgtgctcacctcttca

SEQ ID NO: 35 - DNA, 22 bp, Homo sapiens
taggccaagtttgtatcaggtg

SEQ ID NO: 36 - DNA, 22 bp, Homo sapiens
gtgtggcatttgttttgtgtaa

SEQ ID NO: 37 - DNA, 20 bp, Homo sapiens
agctcatcttcgccatcgtg

SEQ ID NO: 38 - DNA, 20 bp, Homo sapiens
accgggttaaagcagcagtg

(19) United States
(12) Patent Application Publication
(10) Pub. No.: US 2007/0020230 A1
(43) Pub. Date: Jan. 25, 2007

(54) Use of chemokines, and pharmaceutical preparations containing the same

(75) Inventors: Christian Kaps, Berlin (DE); Jochen Ringe, Horum Neuendorf (DE)

Correspondence Address: SIEBERTH & PATTY, LLC, 4703 BLUEBONNET BLVD, BATON ROUGE, LA, 70809, US

(73) Assignee: Trans Tissue Tochologies GmbH

(21) Appl. No.: 10/565,226

(22) PCT Filed: Jul. 9, 2004
(86) PCT No.: PCT/EP04/07581

§ 371 Date: Jul. 10, 2006

(30) Foreign Application Priority Data
Jul. 21, 2003 (DE) 103 33 901.9

(51) Int. Cl. A61K 48/00; A61K 38/19

(52) U.S. Cl. 424/85.1; 514/44

(57) ABSTRACT

The present invention relates to the use of chemokines and/or nucleic acids encoding a chemokine for recruiting mesenchymal precursor and/or stem cells in vivo and in vitro. The present invention also relates to pharmaceutical preparations which comprise these substances and which are preferably intended for recruiting mesenchymal precursor and/or stem cells for tissue synthesis.

The present invention relates to the use of chemokines and/or nucleic acids encoding, a chemokine for recruiting mesenchymal precursor and/or stem cells in vivo and in vitro. The present invention also relates to pharmaceutical preparations which comprise these substances and which are preferably intended for recruiting mesenchymal precursor and/or stem cells for tissue synthesis.

### FIELD OF THE INVENTION

Osteoarthritis is the most frequently occurring joint disease worldwide. During the course of this primarily degenerative joint disease, there is a stepwise local destruction of the joint surface, i.e. degeneration of the articular cartilage. The consequences of this are pain and restriction of function and mobility. Some of the factors which influence the development of osteoarthritis are age, sex, weight, osteoporosis, mechanical overstraining, incorrect positions and traumas.

Conventional orthopedic treatment methods such as “debridement”, “joint shaving”, “microfracture” and “drilling” are frequently only insufficiently effective. All that frequently remains as a last resort is a reconstructive intervention involving an endoprosthetic joint replacement. Alternative methods for restoring joint cartilage, or bones use the techniques of tissue engineering, i.e. artificial tissue growth. For this, autologous cartilage cells or mesenchymal precursor or stem cells are removed from the patient and propagated in elaborate cell culture methods. In a second operation, these cells are injected into the defective region which is covered with a periosteal flap (ACT, autologous chondrocyte transplantation) or introduced into the defective region after having been packed in three-dimensional biomaterials which promote cartilage maturation (chondrogenesis) or bone maturation (osteogenesis) [see also U.S. Pat. No. 5,891,455].

By contrast, more recent methods are directed toward regenerating defects directly in tissue, i.e. in-situ regeneration. For this, biomaterials which are provided with biologically active factors such as growth and differentiation factors, adhesion molecules, extracellular matrix molecules and chemotactic factors, are introduced into the defective region in order to direct mesenchymal cells to the site of the defect and to stimulate regeneration of the defective tissue at this site.

Proteins which possess the property of supporting human cells during migration, or of stimulating these cells to migrate, are termed chemotactic factors. These factors are, for example, extracellular matrix molecules and secreted proteins which diffuse from the tissue. Chemotactic factors comprise a number of proteins such as growth and differentiation factors (for example from the transforming growth factor (TGF) family, the bone morphogenetic protein (BMP) family, the cartilage-derived morphogenetic proteins (CDMP), from the fibroblast growth factor (FGF) family, the connective tissue growth factor (CTGF), from the platelet-derived growth factor (PDGF) family, from the vascular endothelial growth factor (VEGF) family), or from the epidermal growth factor (EGF) family), extracellular matrix molecules (for example osteopontin, fibronectin, hyaluronic acid, heparin, thrombospondin, collagens and vitronectin) and chemokines (CCL, CXCL, CX₃CL and XCL).

The use of extracellular matrix molecules (osteopontin) and secreted growth and differentiation factors (cartilage-derived morphogenetic protein) as chemotactic factors which induce mesenchymal cells not only to migrate into the defective region but also, at the same time, to mature in a tissue-specific manner is described in DE 199 57 388A. Matrix molecules do not diffuse in the tissue and are therefore only suitable for being used as demotactic factors under certain circumstances. Some of the secreted proteins adhere to matrix proteins, with this in turn restricting their freedom of movement. However, they also have a differentiating effect. If the differentiation takes place too early, the tissue is not formed at the desired site. In addition, it is not possible to uncouple recruitment and differentiation. The choice of the chemotactic factor also determines the differentiation process.

The methods which have been used thus far therefore first of all require the isolation of autologous tissue-forming cells which have to be implanted in the patient at the site at which new tissue (usually cartilage or bone) is to be resynthesized. However, the isolation of autologous cells is time-consuming and is associated, as far as the patient is concerned, with at least one prior biopsy, if not an operation, for obtaining the cell material.

### SUMMARY OF THE INVENTION

In a first embodiment, the present invention relates to the use of a chemokine and/or of a chemokine-encoding nucleic acid for producing a pharmaceutical preparation. The pharmaceutical preparation is preferably intended for recruiting mesenchymal, preferably local mesenchymal precursor cells, preferably from the bone marrow, for tissue synthesis.

In a second alternative embodiment, the invention relates to the use of a chemokine and/or of a chemokine-encoding nucleic acid for recruiting mesenchymal, preferably local mesenchymal precursor cells from the bone marrow in vitro.

The chemokine is preferably selected from the group consisting of CCL19, CCL21, CCL27, CCL28, CCL20, CXCL9, CXCL10, CXCL11, CXCL16, CXCL13, CXCL5, CXCL6, CXCL8, CXCL12, CCL2, CCL8, CCL13, CCL25, CCL3, CCL4, CCL5, CCL7, CCL14, CCL15, CCL16, CCL23, CX₃CL1, XCL1, XCL2, CCL1, CCL17, CCL22, CCL11, CCL24, CCL26, CXCL1, CXCL2, CXCL3 and CXCL7, more preferably from the group consisting of CCL19, CCL21, CCL27, CCL28, CCL20, CXCL9, CXCL10, CXCL11, CXCL16, CXCL13 and CXCL5, CXCL6, CXCL8, CXCL12, CCL2, CCL8, CCL13 and CCL25, most preferably from the group consisting of CCL19, CCL21, CCL27, CCL28, CCL20, CXCL9, CXCL10 and CXCL11.

It is possible to use a chemokine or a mixture of chemokines. Alternatively, it is possible to use a chemokine fragment or a chemokine derivative which possesses the ability to bind to a chemokine receptor. In each case, the chemokine can be a natural chemokine or a synthetic chemokine.

The nucleic acid which encodes a chemokine can be present in the form of RNA, DNA, cDNA or ssDNA and can be of natural or synthetic origin.

The pharmaceutical preparation is preferably present in a form which is suitable for injection. The preparation can additionally comprise:
one or more suitable auxiliary substances;.
one or more biologically degradable polymers;
at least one active compound which is selected from differentiation and growth factors and mixtures thereof, with the differentiation and growth factors preferably inducing chondrogenesis or osteogenesis,
   and mixtures of 2 or more of the above.

In a third embodiment, the invention relates to a pharmaceutical preparation which comprises a chemokine as defined above.

In a fourth embodiment, the invention finally relates to a pharmaceutical preparation which comprises a nucleic acid as defined above.

### BRIEF DESCRIPTION OF THE FIGURES

FIG. 1: Using RT-PCR to detect the expression of the chemokine receptors in human mesenchymal stem cells.

FIG. 2: Detecting dose-dependent stem cell migration as a reaction to CXCL12.

### PRECISE DESCRIPTION OF THE INVENTION

According to the invention, proteins of the chemokine family can be used for recruiting mesenchymal precursor cells, in particular mesenchymal stem cells, for example from the bone marrow, with the recruitment being able to take place in vivo and in vitro. The recruitment can be used therapeutically in connection with curing tissue defects, in particular pathogenic and/or traumatic and/or age-associated cartilage defects, cartilage lesions, bone defects and bone fractures.

The chemokine(s) is/are made available at a particular site. Emanating from this site, a concentration gradient is created due to diffusion. Due to this concentration gradient, the mesenchymal cells are directed to the given site, with this being referred to as recruitment. The cells receive the appropriate stimulus as a result of the chemokines binding to specific chemokine receptors.

The present invention is based on the insight that human or animal mesenchymal precursor cells and stem cells possess corresponding receptors. The expression or the presence of these receptors in human or animal mesenchymal precursor cells and stem cells has not previously been reported in the scientific literature and is substantiated in this present document.

Without wishing to be bound to this, it is assumed that the mesenchymal precursor cells and stem cells react to chemokines precisely because of the expression of these receptors and can consequently migrate due to the chemokine signal. In this connection, the response behavior and the migration rate presumably depend on the level at which the receptor is expressed on the given cell. The ligands of the receptors which are expressed to the highest extent are therefore presumably the chemokines to which the mesenchymal precursor cells and stem cells respond most strongly.

As the level of expression declines, so does the likelihood that the cells will react chemotactically to the chemokines corresponding, to the chemokine receptor, and migrate. The migration properties of the precursor cells and stem cells, and the “attraction” potential of the chemokines, are used in accordance with the invention in order to recruit, in situ, mesenchymal, preferably even local, precursor and stem cells to a specific site, for example to the site of a defect (e.g. a cartilage lesion).

Chemokines are proteins (5-20 kDa) which play an important physiological role in a large number of processes such as the hematopoiesis of blood stem cells and the chemotaxis of leukocytes. Chemotaxis is understood as being the positive or negative movement reaction, which is induced by a chemical stimulus and takes place in the direction toward the stimulus or in the direction away from it, of mobile organisms or cells whose cell membrane is activated by corresponding “chemotactic substances” (chemokines or chemotaxins). This activation is mediated by a corresponding cell surface receptor (chemokine receptor) to which the chemokine binds. In the context of the present invention, the induced chemotaxis of specific target cells, which is targeted toward a defective site, is also termed “recruitment”.

The amino acid sequences of all the chemokines are similar and characterized by a constant arrangement of four cysteines. The chemokine family is subdivided into four subfamilies, i.e. CC, CXC, CX₃C and C chemokines, depending on the location of the first two cysteines, with the representatives of the C subfamily only possessing two cysteines (see Table 1 below). A detailed account can be found in Murphy et al. (2000) “International union of pharmacology, XXII, Nomenclature of chemokine receptors”, Pharmacol Rev 52: 145-176, which is hereby incorporated herein by reference. In that which follows, the nomenclature described by Murphy et al. is used for designating preferred chemokines which are to be used in accordance with the invention. The chemokines themselves are designated CCL, CXCL, CX₃CL and XCL. In these designations, “L” stands for ligand. In addition to the nomenclature names, trivial names are also frequently used in the literature.

The chemokines and their receptors are expressed by a large number of hematopoietic and nonhematopoietic cells. The chemokine activity is initiated by binding to a specific G protein-coupled receptor. Although most investigations regarding the mode of action of chemokines have thus far been carried out on leukocytes, the function of the chemokines extends far beyond leukocyte physiology.

Chemokine receptors are classified as receptors for CCL, CXCL, CX₃CL and XCL and are systematically designated CCR, CXCR, CX₃CR and XCR (“R” stands for receptor) (see Table 1 below). Some of them can bind several chemokines in a subfamily. The amino acid sequences of the chemokine receptors are 25-80% identical with each other and 25% identical with many other G protein-coupled receptors [Murphy et al. (2000) “International union of pharmacology, XXII, Nomenclature of chemokine receptors”, Pharmacol Rev 52: 145-176].

The N terminus is located on the extracellular side of the membrane and usually glycosylated while the C terminus is located on the cytoplasmic side and is phosphorylated. Three extracellular loops alternate with three intracellular loops and link seven hydrophobic transmembrane domains. A two-step model for the receptor activation has been developed: the binding of the chemokine to the receptor first of all leads to a conformational change in the chemokine after which the receptor is activated by the N terminus of the chemokine. In connection with this, GDP which is bound to the α subunit of the G protein is replaced with GTP. The G protein dissociates from the receptor and triggers a cascade of biochemical reactions in the cytoplasmic space.

CC and CXC receptors have been detected in monocytes, lymphocytes, basophilic and eosinophilic granulocytes and chondrocytes. Eleven CC receptors (CCR1-CCR11) belong to the CC chemokine receptor family. They possess seven characteristic sequence segments which differentiate them from the 6 receptors of the CXCR family (CXCR1-CXCR6).

TABLE 1
Human chemokine receptors and their ligands

| Chemokine | |
|---|---|
| receptor | Chemokine ligand |
| CCR1 | CCL3, CCL4, CCL5, CCL7, CCL8, CCL13, |
| | CCL14a, CCL14b, CCL15, CCL16, CCL23 |
| CCR2 | CCL2, CCL7, CCL8, CCL13 |
| CCR3 | CCL5, CCL7, CCL8, CCL11, CCL13, |
| | CCL14, CCL15, CCL24, CCL26 |
| CCR4 | CCL3, CCL5, CCL7, CCL22 |
| CCR5 | CCL3, CCL4, CCL5, CCL8, CCL11, CCL13, CCL14 |
| CCR6 | CCL20 |
| CCR7 | CCL19, CCL21 |
| CCR8 | CCL1, CCL16 |
| CCR9 | CCL25 |
| CCR10 | CCL27, CCL28 |
| CCR11 | CCL2, CCL8, CCL13, CCL19, CCL21, CCL25 |
| CXCR1 | CXCL5, CXCL6, CXCL8 |
| CXCR2 | CXCL1, CXCL2, CXCL3, CXCL5, CXCL7, CXCL8 |
| CXCR3 | CXCL9, CXCL10, CXCL11 |
| CXCR4 | CXCL12 |
| CXCR5 | CXCL13 |
| CXCR6 | CXCL16 |
| XCR1 | XCL1, XCL2 |
| CX3CR1 | CX3CL1 |

In their investigations, the inventors have observed that there is a gradation in the expression of the different chemokine receptors on mesenchymal cells. This gradation is depicted in Table 3 which is presented below. This in turn gives rise to the preferred employment, within the context of the use according to the invention, of the chemokines which bind to the receptors which are most frequently expressed.

Preference is given to using the chemokines having the numbers 1-39 in Table 4, preferably the numbers 1-18, and particular preference is given to those having the numbers 1-8. These chemokines can be used in the form of the chemokines or of their fragments and/or derivatives, or else in the form of a nucleic acid (for example DNA, cDNA, RNA or ssDNA) which encodes a chemokine. According to the invention, a fragment of a, chemokine is understood as being a peptide which comprises a constituent sequence of the amino acid sequence of the chemokine. According to the invention, a derivative of a chemokine is understood as being a peptide or protein having an amino acid sequence which is derived by deletion, substitution, addition or point mutation from the amino acid sequence of a chemokine. Of fundamental importance for fragments and/or derivatives to be suitable is the retention of the ability to bind to the chemokine receptor and, preferably retention of the binding specificity as well.

A pharmaceutical preparation which comprises the chemokine and/or a nucleic acid encoding the chemokine is produced for the diagnostic and/or therapeutic use using conventional methods. The pharmaceutical preparation is preferably intended for injection. Suitable methods for producing pharmaceutical preparations which comprise proteins and nucleic acids, and auxiliary substances which are suitable for this purpose, are known and will not be described here. It is within the ability of the skilled person to design such a preparation. Injection solutions, fibrin adhesives, substrates for transplantation, matrices, tissue patches or suture materials are, for example, suitable.

For application, the preparation is now introduced, preferably by means of injection or using a fibrin adhesive, a substrate, a matrix or a patch, into the tissue defect such as a bone defect or cartilage defect. Examples of suitable substrates are disclosed in DE 199 57 388, which is hereby incorporated herein by reference. A connection to the bone marrow space can be created for the purpose of attracting mesenchymal precursor and/or stem cells. After the mesenchymal cells have migrated into the bone or cartilage defect, they synthesize, in the defect region, regeneration tissue which fills in and stabilizes the defect region. The synthesis of the bony or cartilaginous regenerated tissue can be supported by admixing growth and differentiation factors which promote osteogenesis or chondrogenesis.

The invention consequently preferably relates to the use of chemokines for producing pharmaceutical preparations for recruiting local mesenchymal precursor cells from the bone marrow for regenerating diseased or traumatic joint defects, predominantly in connection with arthritis.

Within the meaning of the present invention, mesenchymal precursor cells and stem cells are cells which possess the property of developing into one or more mesenchymal tissues. The examples which may be mentioned are cartilage using chondrocytes, bone using osteocytes, tendons using tenocytes, ligaments using tenocytes, cardiac muscle using cardiomyocytes, connective tissue using fibroblasts, fibrous tissue using fibroblastic cells and neuronal tissue using astrocytes and neurons. The precursor cells can consequently be precursor cells of chondrocytes, osteocytes, tenocytes, cardiomyocytes, fibroblasts, fibroblastic cells, astrocytes or neurons. Consequently, the precursor cells can, for example, be precursor cells/stem cells of cartilage cells, which precursor cells develop exclusively into cartilage cells or else precursor cells which possess the ability to develop into cartilage cells and bone cells or else precursor cells which possess the ability to develop exclusively into bone cells.

During use of the preparation, the chemokines which are present in the preparation “attract” the mesenchymal precursor cells from the surrounding tissue in the vicinity of the joint, preferably from the bone marrow, and direct them to the defective site. The mesenchymal precursor cells then remain at this site and form bony regeneration tissue in the bone defect and cartilaginous regeneration tissue in the cartilaginous defect. A similar attraction can naturally also be used for culturing corresponding cells, for example derived from biopsies, in vitro.

In a preferred embodiment, the invention relates to the use of chemokines for recruiting mesenchymal stem cells. Within the meaning of the present invention, mesenchymal stem cells are mesenchymal precursor cells which possess the ability to develop into several, at least two, different mesenchymal tissues.

In another preferred embodiment, the present invention relates to the use of chemokines for recruiting mesenchymal precursor cells or stem cells from the bone marrow. For this, small channels are drilled arthroscopically from the defective site in the cartilage into the bone tissue underlying the cartilage such that a connection is formed between the defective site and the bone marrow. Introducing chemokines into the defective site then attracts mesenchymal precursor or stem cells, which colonize the defective site and, at this site, form regeneration tissue which closes the defect.

Alternatively, it is possible to envisage using nucleic acids which encode a chemokine. In this connection, it is advantageous to introduce RNA, DNA, cDNA or ssDNA which is taken up by local cells, read and expressed as mature protein.

In another preferred embodiment, the chemokines which are used for recruiting mesenchymal precursor cells are mixed with biologically degradable polymers or biomaterials. Within the meaning of the invention, biologically degradable polymers are those, preferably three-dimensional, polymer structures which do not exert any toxic effects on cells, which do not induce any immune reaction and which promote the synthesis of cartilage or bone tissue. The introduction of biologically degradable polymers together with chemokines into the defective site to be closed leads to the attraction of mesenchymal precursor cells, which migrate directly into the polymer tissue, where they find a three-dimensional polymer structure for optimal tissue maturation into cartilage or bone. Examples of these polymers or biomaterials are polylactide, polyglycolide, poly(lactide-glycolide), polylysine, polycaprolactone, alginate, agarose, fibrin, hyaluronic acid, polysaccharides, cellulose, collagens and hydroxylappatite.

The chemokines can also be used jointly with growth and differentiation factors in the same preparation (or else administered in separate preparations). Very particular preference is given to chemokine, polymer and growth and differentiation factors being used jointly. Introducing such a mixture into the defective site has the advantage that, in addition to the optimal polymer structure which is already promoting tissue maturation, the attracted mesenchymal precursor cells are also additionally stimulated to mature into tissue by growth and differentiation factors.

In a preferred embodiment, the present invention relates to the use of chemokines together with growth and differentiation factors which induce cartilage maturation. Within the meaning of the present invention, factors which induce cartilage maturation are growth and differentiation factors which, from the point of view of developmental biology, stimulate a precursor cell to differentiate and mature into a chondrocytic cell type or a mature cartilage cell for producing cartilage matrix. The use of members of the cartilage-derived morphogenetic protein (CDMP) and bone morphogenetic protein (BMP) family, as well as insulin, is advantageous in this connection.

In another preferred embodiment, the present invention relates to the use of chemokines together with growth and differentiation factors which induce bone maturation. Within the meaning of the present invention, factors which induce bone maturation are growth and differentiation factors which, from the developmental biology point of view, stimulate a precursor cell to differentiate and mature into a bony cell type or a mature bone cell for producing bone matrix. The use of members of the bone morphogenetic protein (BMP) family, particularly preferably the members BMP-2 and BMP-7, is advantageous in this connection.

The following examples are intended to illustrate the invention. However, they are not intended to limit the invention.

### EXAMPLES

### Example 1

Isolating and Culturing Human Mesenchymal Stem Cells

Human mesenchymal stem cells (MSCs) were isolated as follows using a previously described protocol for obtaining MSCs from the bone marrow.

At most 3 ml of bone marrow punctate are mixed with 10 ml of PBS and centrifuged for 10 min at 310 g at room temperature. The cell pellet is resuspended and once again washed with PBS (8000 mg of NaCl/l 200 mg of KCl/l, 1150 mg of Na₂HPO₄/l, 200 mg of KH₂PO₄/l). The cells are taken up in 20 ml of DME medium (containing 10-20% FBS, 2% HEPES, 4 mM L-glutamine, 100 U of penicillin/ml, 100 μg of streptomycin/ml). In each case, 5 ml of this cell suspension are loaded onto 20-ml of a Percoll density gradient having a density of 1.073 g/ml. The cells are centrifuged at 900 g for 32 min.

The upper phase is transferred to a new centrifuge tube. After 2.5 times the volume of PBS has been added, the mixture is centrifuged once again at 310 g for 6 minutes. The cell pellet is taken up in DME medium.

1.5×10⁵-3.5×10⁵ cells/cm² are added, for culturing, to a cell culture flask and incubated, at 37° C. and 5% CO₂, in DME medium (Biochrom AG, Berlin, Catalogue No. FG0415, Dulbecco's modified Eagle medium containing 3.7 g of NaHCO₃/l and 1.0 g of D-glucose/l). The medium is changed for the first time after 72 hours and then every 3-4 days. The cells which have been isolated in this way grow confluent after 2-3 weeks and are then transferred, by means of trypsinization, into a new culture vessel at a cell density of 6000 cells/cm² of culture surface (passage 1). After about a week, the cells are trypsinized once again (passage 2).

The homogeneity of the culture of human mesenchymal stem cells which is obtained is verified by means of FACS analysis, in connection with which it is necessary to detect the surface antigens endoglin and ALCAM and not to detect the surface antigens CD34, CD45 and CD14. This was confirmed.

### Example 2

Analyzing Gene Expression for Detecting the Chemokine Receptors

The isolated, expanded and verified human mesenchymal stem cells express chemokine receptors. This was demonstrated for several human patients (n=3) by means of RT-PCR as follows:

a. Isolating the Total RNA

Tri Reagent LS™ is used for isolating the total RNA. The MSCs are cultured to confluence. After the cell culture medium has been discarded, the cell lawn is overlaid with 0.4 ml of Tri Reagent LS™ per 10 cm² of growth area in order to lyse the cells. The lysate is transferred to a sterile reaction vessel and incubated at room temperature (RT) for 5 minutes. The lysate is treated with 0.1 ml of bromochloropropane (BCP) per 0.75 ml of Tri Reagent LS™, after which it is shaken for 15 seconds and incubated at RT for 10 minutes. A subsequent centrifugation for 15 minutes at 4° C. and 12 000 g results in phase separation. The aqueous phase is taken off in 200 μl aliquots and transferred to a reaction vessel. The RNA solution is treated with 0.5 ml of isopropanol per 0.75 ml of Tri Reagent LS™ and left at −20° C. for at least 7 minutes. The precipitated RNA is pelleted by centrifuging for 8 minutes at 4° C. and 12 000 g. The resulting RNA pellet is washed with 70% EtOH, dried in air and taken up in 20 μl of DEPC-H₂O. In order to dissolve the pellet, it is heated at 55° C. for 10 minutes. The content of isolated total RNA is determined by means of photometric measurement.

b. cDNA Synthesis:

For the cDNA synthesis, 5 μg of total RNA are used in 10 μl of DEPC-H₂O and this solution is treated with 1 μl of oligo-(dT)12-18 primers (in each case one upper and one lower primer as specified in Table 2), in order then to be denatured at 70° C. for 10 min. After the denaturation, the reaction mixture is stored on ice and treated with 4 μl of 5× buffer (0.25 M Tris/HCl, pH 8.3; 0.375 M KCl; 15 MM MgCl₂), 2 μl of 0.1 M DTT, 1 μl of dNTP (in each case 10 mM) and 0.4 μl of RNase inhibitor. After an incubation time of 2 min at 37° C., 1 μl of SuperScript™ reverse transcriptase is then added to the reaction mixture, which is then incubated at 37° C. for a further 60 minutes. After 40 μl of TE (10/1, pH 7.8) have been added, the enzyme is inactivated at 92° C. for 10 min. 2.0 μl of cDNA are used for the RT-PCR reactions.

As standard, 1 μl of cDNA is used per PCR reaction. 2 μl of 10× PCR buffer, 2 μl of 25 mM MgCl₂, 0.2 μl of 10 mM dNTPs, 1 μl of 5 nM primer (Table 2) and 0.5 U of Taq DNA polymerase are added to the cDNA in a PCR reaction vessel and the mixture is made up to a final volume of 20 μl with H₂O. A standard reaction cycle starts with a denaturation at 95° C. for 1 min, with this being followed by hybridization of the primers for 15 sec. at a temperature (Tₐₙ) which is specific for the primers, and a DNA synthesis reaction at 72° C. for 15 sec. This cycle is repeated a total of 35 times. In conclusion, the mixture is kept at 72° C. for 3 min. The PCR products are fractionated by gel electrophoresis. The DNA fragments were eluted from the gel and cloned into the vector pGEM-T Easy (Promega). Following amplification in E. coli, the corresponding plasmid was isolated and sequenced, in order to demonstrate that the corresponding chemokine receptors were amplified by means of the oligonucleotides used in Table 2, with this being confirmed by comparison with the known sequence.

TABLE 2
Oligonucleotides for detecting the expression of human chemokine receptors

| | EMBL | Amplificate | |
|---|---|---|---|
| | nucleotide sequence | length | Oligonucleotide sequence |
| Receptor | database identifier | (base pairs) | (5′ > 3′) |
| ccr1 upper | NM 001295 | 129 | GAGCCAATCAGTAGCCAGCATCT |
| ccr1 lower | NM 001295 | | GTTCCCCCATTTCTATTTCTCGTT |
| ccr2 upper | NM 000647 | 173 | CTCCCTGAAGTAAGCAAAGAC |
| ccr2 lower | NM 000647 | | CCATGTGGCCTGAAAGTAG |
| ccr3 upper | NM 178329 | 148 | GGCAGATACATCCCATTCCTTC |
| ccr3 lower | NM 178329 | | GGTTGCTTCATCTCCTTGGTCCTT |
| ccr4 upper | X85740 | 91 | CAGGGGCCTTTTTGTGCTC |
| ccr4 lower | X85740 | | CATGGTGGACTGCGTGTAAGAT |
| ccr5 upper | NM 000579 | 160 | AGGAGGGAGGTATTCGTAAGG |
| ccr5 lower | NM 000579 | | TTCAAGGGTTTCTCCAATCTG |
| ccr6 upper | NM 031409 | 86 | TGGTTACAGCACAAAATGATGG |
| ccr6 lower | NM 031409 | | TTGCCTAAAATGAGTGATGTGTTG |
| ccr7 upper | NM 001838 | 194 | GCCGCCCTAAAAGCACACTCATCC |
| ccr7 lower | NM 001838 | | TTCCCTTGTCCTCTCCTCCCATCC |
| ccr8 upper | NM 005201 | 198 | TGCAGCCAAATCTTCAACTACC |
| ccr8 lower | NM 005201 | | AAACCTTTCACACCCACACCTT |
| ccr9 upper | NM 031200 | 151 | AGCCTTGGCCCTGTTGTA |
| ccr9 lower | NM 031200 | | TGCCCATATCTGCTCACTGTA |
| ccr10 upper | NM 016602 | 118 | GCCCCGCCTTTCTTCCTGCTCA |
| ccr10 lower | NM 016602 | | CCACCTACTCCCCTTTCCCACGAC |
| ccr11 upper | NM 016557 | 90 | CTCTGCCTTTTGCTTGGATACATA |
| ccr11 lower | NM 016557 | | CACGGCGTCTGAGATTTGAGTT |
| cxcr1 upper | NM 000634 | 177 | CCGTGCTTGTCCCTGTGG |
| cxcr1 lower | NM 000634 | | CTGTGCCTCAAGAGACTGTTC |
| cxcr2 upper | NM 001557 | 146 | AGTTTATGATTCCACCTACA |
| cxcr2 lower | NM 001557 | | TTCAACATCCTAAACATAAA |
| cxcr3 upper | NM 001504 | 140 | GTGGCCGAGAAAGCAGGGTAGACG |
| cxcr3 lower | NM 001504 | | CAGGCGCAAGAGCAGCATCCACAT |
| cxcr4 upper | NM 003467 | 141 | GATCCCTGCCCTCCTGCTGACTAT |
| cxcr4 lower | NM 003467 | | AGGCCAACCATGATGTGCTGAAAC |
| cxcr5 upper | NM 032966 | 170 | CCGGATCCTGGGTGGTCTG |
| cxcr5 lower | NM 032966 | | CCGCCGGGTTTGATTGAT |
| cxcr6 upper | NM 006564 | 119 | GACTTTCCTTCCTCCATCTCCA |
| cxcr6 lower | NM 006564 | | GGCCGTGCTCACCTCTTCA |
| Cx3cr upper | NM 001337 | 169 | TAGGCCAAGTTTGTATCAGGTG |
| Cx3cr lower | NM 001337 | | GTGTGGCATTTGTTTTGTGTAA |
| xcr upper | NM 005283 | 181 | AGCTCATCTTCGCCATCGTG |
| xcr lower | NM 005283 | | ACCGGGTTAAAGCAGCAGTG |

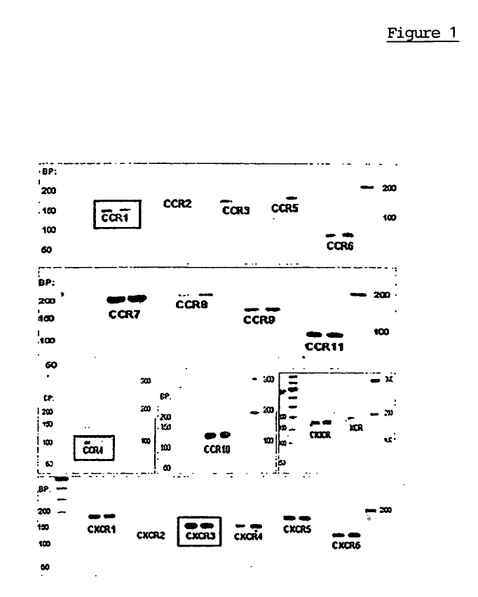

The expression analyses, which were carried out for several patients (n=3), of human bone marrow mesenchymal stem cells with regard to the presence of human chemokine receptors (FIG. 1) showed high expression of receptors 1-9, medium expression of receptors 10-17 and weak expression of receptors 18-19 (Table 3).

TABLE 3
Expression, and level of expression, of chemokine
receptors in human mesenchymal stem cells

| Order of expression | | |
|---|---|---|
| level | Receptor | Ligands |
| 1 | CCR7 | CCL19, CCL21 |
| 2 | CCR10 | CCL27, CCL28 |
| 3 | CCR6 | CCL20 |
| 4 | CXCR3 | CXCL9, CXCL10, CXCL11 |
| 5 | CXCR6 | CXCL16 |
| 6 | CXCR5 | CXCL13 |
| 7 | CXCR1 | CXCL5, CXCL6, CXCL8 |
| 8 | CXCR4 | CXCL12 |
| 9 | CCR11 | CCL2, CCL8, CCL13, CCL19, |
| | | CCL21, CCL25 |
| 10 | CCR1 | CCL3, CCL4, CCL5, CCL7, |
| | | CCL8, CCL13, CCL14a, CCL14b, |
| | | CCL15, CCL16, CCL23 |
| 11 | CCR9 | CCL25 |
| 12 | CX3CR | CX3CL1 |
| 13 | XCR | XCL1, XCL2 |
| 14 | CCR8 | CCL1, CCL16 |
| 15 | CCR4 | CCL3, CCL5, CCL17, CCL22 |
| 16 | CCR5 | CCL3, CCL4, CCL5, CCL8, |
| | | CCL11, CCL13, CCL14 |
| 17 | CCR3 | CCL5, CCL7, CCL8, CCL11, |
| | | CCL13, CCL14, CCL15, CCL24, |
| | | CCL26 |
| 18 | CXCR2 | CXCL1, CXCL2, CXCL3, CXCL5, |
| | | CXCL7, CXCL8 |
| 19 | CCR2 | CCL2, CCL7, CCL8, CCL13 |

The differing levels of expression suggest that, in this connection, ligands of the receptors which are expressed at the highest level are those chemokines to which the mesenchymal stem cells respond most strongly, and migrate. As the level of expression declines, so does the likelihood that the stem cells react chemotactically to the chemokines which correspond to the chemokine receptor, and migrate. Based on this, it follows that human mesenchymal stem cells are activated, and can be recruited in situ, most strongly by stimulation with chemokine No. 1, with this effect declining down to chemokine No. 39, in Table 4.

TABLE 4
Chemokines for the in situ recruitment
of mesenchymal precursor cells

| | | Reference sequence (EMBL | |
|---|---|---|---|
| No. | Chemokine | nucleotide sequence database) | Trivial name |
| 1 | CCL19 | NM 006274 | MIP-3β |
| 2 | CCL21 | NM 002989 | 6Ckine |
| 3 | CCL27 | NM 006664 | CTACK |
| 4 | CCL28 | NM 148672 | MEC |
| 5 | CCL20 | NM 004591 | MIP-3α |
| 6 | CXCL9 | NM 002416 | Mig |
| 7 | CXCL10 | NM 001565 | IP-10 |
| 8 | CXCL11 | NM 005409 | 1-TAC |
| 9 | CXCL16 | NM 022059 | |
| 10 | CXCL13 | NM 006419 | BCA-1 |
| 11 | CXCL5 | NM 002994 | ENA-78 |
| 12 | CXCL6 | NM 002993 | GCP-2 |
| 13 | CXCL8 | NM 000584 | IL-8 |
| 14 | CXCL12 | NM 000609 | SDF-1α |
| 15 | CCL3 | NM 002983 | MIP-1α |
| 16 | CCL4 | NM 002984 | MIP-1β |
| 17 | CCL5 | NM 002985 | RANTES |
| 18 | CCL7 | NM 006273 | MCP-3 |
| 19 | CCL8 | NM 005623 | MCP-2 |
| 20 | CCL13 | NM 005408 | MCP-4 |
| 21 | CCL14 | NM 004166 | HCC-1 |
| 22 | CCL15 | NM 004167 | HCC-2 |
| 23 | CCL16 | NM 004590 | HCC-4 |
| 24 | CCL23 | NM 005064 | MPIF-1 |
| 25 | CCL25 | NM 005624 | TECK |
| 26 | CX3CL1 | NM 002996 | Fractalkine |
| 27 | XCL1 | NM 002995 | Lymphotactin |
| 28 | XCL2 | NM 003175 | SCM-1β |
| 29 | CCL1 | NM 002981 | I-309 |
| 30 | CCL17 | NM 002987 | TARC |
| 31 | CCL22 | NM 002990 | MDC |
| 32 | CCL11 | NM 002986 | Eotaxin |
| 33 | CCL24 | NM 002991 | Eotaxin-2 |
| 34 | CCL26 | NM 006072 | Eotaxin-3 |
| 35 | CXCL1 | NM 001511 | GROα |
| 36 | CXCL2 | NM 002089 | GROβ |
| 37 | CXCL3 | NM 002090 | GROγ |
| 38 | CXCL7 | NM 002704 | NAP-2 |
| 39 | CCL2 | NM 002982 | MCP-1 |

### Example 3

In order to treat a joint surface which is markedly deformed arthritically, small communication channels are first of all prepared between the bone marrow space and the joint cavity by means of drilling a number of fine bore holes (1-2 mm). After that, a wool-like polymer construct (polyglycolide), combined with hyaluronic acid and chemotactically acting chemokine (CCL19), is glued, and fitted, over the joint surface using fibrin or acrylic adhesive.

### EXAMPLE 4

In order to treat the joint surface from Example 3 having a defect size of 6 cm², 1.2 ml of fibrin adhesive together with 1000 ng of growth factor (cartilage-derived morphogenetic protein) and 2000 ng of chemokine (CXCL9) are introduced into the cartilage defect, after the apertures into the marrow space have been prepared, and solidified by simultaneously adding 100 μl of thrombin.

### EXAMPLE 5

Chemotactic activity of the chemokine CXCL12 (SDF-1α) on bone marrow mesenchymal stem cells

The isolated, expanded and verified human mesenchymal stem cells exhibit a dose-dependent chemotactic activity with regard to the chemokine CXCL12 (SDF-1α), This was demonstrated by means of a 96-multiwell chemotaxis test. The 96-multiwell chemotaxis plates which are used in this test consist of an upper part and lower part of a well which are separated by a permeable polycarbonate membrane (pore diameter, 8 μm). The CXCL12 which is introduced into the lower part generates a concentration gradient across the membrane, activated cells from the upper part of the well migrate into the membrane and into the lower part of the well. The detection is performed as follows:

The cells are first of all cultured in normal DMEM culture medium. About 22 hours before the test, the culture medium is removed and the cells are washed with PBS and kept, until the test, in serum-free diet medium (DME medium, contains 1.0 g of glucose/l, 0.2% bovine serum albumin, 2 mM L-glutamine; 100 U of penicillin/ml; 100 μg of streptomycin/ml). Immediately before beginning the test, the cells are trypsinized and the cell number and vitality are determined and the cells are once again taken up in diet medium. 3×10⁴ cells in 40 μl of diet medium are used per upper well of a 96-well plate.

In order to determine the dose-dependent chemotactic activity of CXCL12 (SDF-1α), different concentrations (1-500 nM) of this latter chemokine are added to the diet medium and 35 μl of this medium are added in triplicate to the lower well. Control mixtures which are used are, in the first place, 3×10⁴ cells in 40 μl of diet medium per upper well and 30 μl of serum-containing culture medium without chemokine in the lower well (positive control) and, in second place, 3×10⁴ cells in 40 μl of diet medium per upper well and 30 μl of diet medium without chemokine in the lower well (negative control). The 96-well chemotaxis plates are incubated at 37° C. and under a 5% CO₂ atmosphere for 20 hours. The upper side of the filter (non-migrated side) is wiped in order to remove non-migrated cells. The cells on the underside of the filter (migrated cells) are fixed for 3 min with ice-cold ethanol/acetone (1:1 v/v) and then stained using the Merck Hemacolor® rapid staining system. The membrane is kept moist and three representative photo fields are counted per well. Prior to this, the distribution of the cells in the given well is assessed at lower magnification.

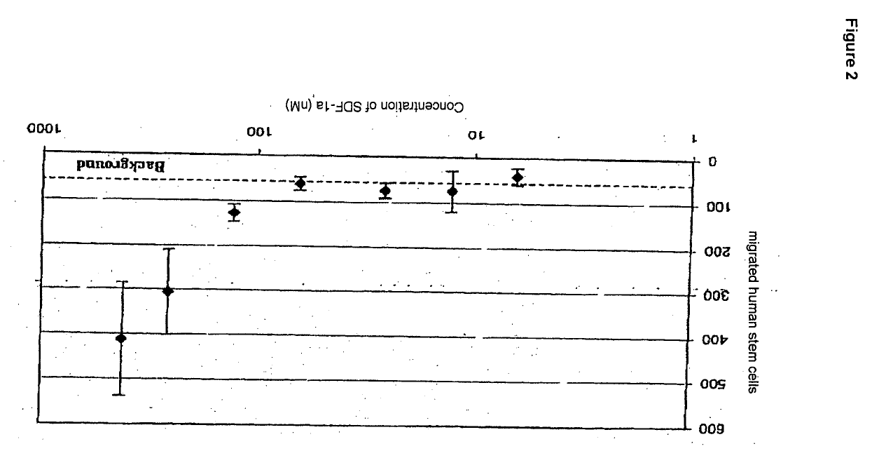

These investigations of human bone marrow mesenchymal stem cells with regard to the chemotactic activity of CXCL12 (SDF-1α) demonstrated that this chemokine has a dose-dependent effect on human mesenchymal stem cells. This is shown in FIG. 2. The highest response of the cells was measured at a concentration of about 500 nM. Below a concentration of somewhat less than 100 nM, the number of migrated cells corresponds approximately to the number of migrated cells in the negative control. This significantly verifies the recruitment effect according to the invention of chemokines on bone marrow mesenchymal precursor cells.

1.-20. (canceled)

21. A method comprising producing a pharmaceutical preparation from at least:
a) a chemokine, or
b) a nucleic acid encoding a chemokine, or
c) a combination of a) and b), or
d) a chemokine fragment which possesses the ability to bind to a chemokine receptor, or
e) a chemokine derivative which possesses the ability to bind to a chemokine receptor.

22. The method as claimed in claim 21 wherein a) or b) or a combination of a) and b) is used in producing the pharmaceutical preparation.

23. The method as claimed in claim 21, wherein said pharmaceutical preparation is capable of recruiting mesenchymal precursor cells and/or mesenchymal stem cells for forming tissues.

24. A method comprising recruiting (i) mesenchymal precursor cells, (ii) local mesenchymal precursor cells and/or (iii) mesenchymal stem cells, with:
a) a chemokine, or
b) a nucleic acid encoding a chemokine, or
c) a combination of a) and b), or
d) a chemokine fragment which possesses the ability to bind to a chemokine receptor, or
e) a chemokine derivative which possesses the ability to bind to a chemokine receptor.

25. The method as claimed in claim 24 wherein (i), (ii), and/or (iii) are recruited with a chemokine, a nucleic acid encoding a chemokine, or a combination of a chemokine and a nucleic acid encoding a chemokine.

26. The method as claimed in any of claims 21, 22, 23, or 24 wherein the chemokine is selected from the group consisting of CCL19, CCL21, CCL27, CCL28, CCL20, CXCL9, CXCL10, CXCL11, CXCL16, CXCL13, CXCL5, CXCL6, CXCL8, CXCL12, CCL2, CCL8, CCL13, CCL25, CCL3, CCL4, CCL5, CCL7, CCL14, CCL15, CCL16, CCL23, CX3CL1, XCL1, XCL2, CCL1, CCL17, CCL22, CCL11, CCL24, CCL26, CXCL1, CXCL2, CXCL3, CXCL7, and mixtures thereof, wherein the chemokine fragment is a fragment of any of the foregoing chemokines, and wherein the chemokine derivative is a derivative of any of the foregoing chemokines.

27. The method as claimed in claim 26, wherein the chemokine is selected from the group consisting of CCL19, CCL21, CCL27, CCL28, CCL20, CXCL9, CXCL10, CXCL11, CXCL16, CXCL13, CXCL5, CXCL6, CXCL8, CXCL12, CCL2, CCL8, CCL13, CCL25, and mixtures thereof, wherein the chemokine fragment is a fragment of any of the foregoing chemokines, and wherein the chemokine derivative is a derivative of any of the foregoing chemokines.

28. The method as claimed in claim 27, wherein the chemokine is selected from the group consisting of CCL19, CCL21, CCL27, CCL28, CCL20, CXCL9, CXCL10, CXCL11, and mixtures thereof, wherein the chemokine fragment is a fragment of any of the foregoing chemokines, and wherein the chemokine derivative is a derivative of any of the foregoing chemokines.

29. The method as claimed in any of claims 21, 22, 23, or 24, wherein a mixture of chemokines is used.

30. The method as claimed in claim 21, wherein the nucleic acid encoding a chemokine is in the form of RNA, DNA, cDNA or ssDNA.

31. The method as claimed in claim 21, wherein the pharmaceutical preparation is formed from a nucleic acid encoding a chemokine, and wherein the nucleic acid encoding the chemokine is in the form of RNA, DNA, cDNA, or ssDNA.

32. The method as claimed in any of claims 23, 24, or 25, wherein the mesenchymal precursor cells or mesenchymal stem cells are recruited from bone marrow.

33. The method as claimed in claim 21, wherein the pharmaceutical preparation is produced in a form which is suitable for injection.

34. The method as claimed in claim 33, wherein the pharmaceutical preparation additionally comprises:
one or more suitable auxiliary substances,
one or more biologically degradable polymers,
at least one active compound which is selected from differentiation and growth factors and mixtures thereof,
and mixtures of two or more of the above.

35. The method as claimed in claim 24, wherein the chemokine and/or a nucleic acid encoding a chemokine is used in combination with an active compound which is selected from differentiation and growth factors and mixtures thereof.

36. The method as claimed in claims 34 or 35, wherein the differentiation and growth factors induce chondrogenesis or osteogenesis.

37. A pharmaceutical preparation which comprises (i) a chemokine or (ii) a nucleic acid encoding a chemokine, wherein the chemokine of (i) or (ii) is:
a) a chemokine selected from the group consisting of CCL19, CCL21, CCL27, CCL28, CCL20, CXCL9, CXCL10, CXCL11, CXCL16, CXCL13, CXCL5, CXCL6, CXCL8, CXCL12, CCL2, CCL8, CCL13, CCL25, CCL3, CCL4, CCL5, CCL7, CCL14, CCL15, CCL16, CCL23, CX3CL1, XCL1, XCL2, CCL1, CCL17, CCL22, CCL11, CCL24, CCL26, CXCL1, CXCL2, CXCL3 and CXCL7; or
b) a chemokine selected from the group consisting of CCL19, CCL21, CCL27, CCL28, CCL20, CXCL9, CXCL10, CXCL11, CXCL16, CXCL13, CXCL5, CXCL6, CXCL8, CXCL12, CCL2, CCL8, CCL13 and CCL25; or
c) a chemokine selected from the group consisting of CCL19, CCL21, CCL27, CCL28, CCL20, CXCL9, CXCL10 and CXCL11; or
d) a mixture of any of the foregoing chemokines; or
e) a chemokine fragment which possesses the ability to bind to a chemokine receptor or a chemokine derivative which possesses the ability to bind to a chemokine receptor.

38. A pharmaceutical preparation which comprises a nucleic acid encoding a chemokine in the form of RNA, DNA, cDNA or ssDNA.

39. The pharmaceutical preparation as claimed in claims 37 or 38 which additionally comprises:
A) one or more suitable auxiliary substances, or
B) one or more biologically degradable polymers, or
C) at least one active compound which is selected from differentiation and growth factors and mixtures thereof, or
D) mixtures of two or more of A), B), C).

40. The pharmaceutical preparation as claimed in claims 37 or 38 which is in the form of an injection solution, of a fibrin adhesive, of a substrate for transplantation, of a matrix, of a tissue patch, or of suture material.

41. In a method of forming new tissue in a subject, the improvement comprising forming said tissue by recruiting (i) mesenchymal precursor cells, (ii) local mesenchymal precursor cells and/or (iii) mesenchymal stem cells using
a) a chemokine, or
b) a nucleic acid encoding a chemokine, or
c) a combination of a) and b), or
d) a chemokine fragment which possesses the ability to bind to a chemokine receptor, or
e) a chemokine derivative which possesses the ability to bind to a chemokine receptor.

42. The improvement as claimed in claim 41 comprising forming said tissue using a), b), or c).

43. The improvement as claimed in claims 41 or 42 wherein the cells of (i), (ii), and/or (iii) are recruited from bone marrow.
